(19) United States
(12) Patent Application Publication
(10) Pub. No.: US 2011/0160635 A1
(43) Pub. Date: Jun. 30, 2011

(54) COTTON BALLS, COTTON SWABS AND COTTON SWAB HOLDER

(76) Inventor: Robert J. Baschnagel, Garden City, NY (US)

(21) Appl. No.: 12/647,051

(22) Filed: Dec. 24, 2009

(51) Int. Cl. A61F 13/40; A61F 13/38; B08B 1/00

(52) U.S. Cl. 604/2; 604/1; 15/104.94

(57) ABSTRACT

A cotton swab and cotton swab holder, which can be used together in a cotton swab holder assembly. The cotton swab having a shaft and a cotton portion disposed on an end of the shaft. The cotton swab holder having a handle portion; and a cotton swab holding portion connected to the handle portion, the cotton swab holding portion having a mechanism for holding the shaft of the cotton swab. Also provided is a cotton ball including: a cotton body; a wall defining a cavity disposed on an interior of the cotton body; and one of a liquid or gel contained in the cavity.

### BACKGROUND

1. Field

The present invention relates generally to cotton products and, more particularly, to cotton balls, cotton swabs and cotton swab holders for applying a liquid or gel contained therein to a surface.

2. Prior Art

Cotton swabs are well known in the art, such as those marketed under the tradename Q-Tip®. Such cotton swabs typically have a pair of cotton portions on one or each side of a slender stick. The stick can be wood, paper or plastic. Cotton swabs have many different medical, household, cosmetic and industrial uses and are often used to apply liquid or gel substances, such as medicaments or solvents after dipping one or more of the cotton portions into the liquid or gel or pouring the same onto the cotton portions. Although cotton swabs have many medical uses, they are generally not recommended for cleaning the ear due to the sensitivity of the inner ear. However, the long slender nature of the cotton swab facilitates its unauthorized use in the ear, leading to many ear injuries, particularly in children.

Cotton balls are also well known in the art and also have many medical, household, cosmetic and industrial uses, such as removing nail polish when used together with nail polish remover. However, application of fluids or gels thereto can be cumbersome, resulting in spillage and/or too much or too little of the fluid or gel being applied to the cotton ball than what is desired for the particular application.

### SUMMARY

Accordingly, a holder for a cotton swab is provided. The holder comprising: a handle portion; and a cotton swab holding portion connected to the handle portion, the cotton swab holding portion having a mechanism for releasably retaining a shaft of the cotton swab.

The handle portion can be an elongated handle portion adapted to be gripped by a hand of a user.

The cotton swab holding portion can be bulbous and have a proximal end connected to a distal end of the handle portion.

The mechanism for releasably retaining the cotton swab can comprise: a threaded shaft having a lumen, a male thread and two or more slits formed in the male thread so as to form threaded portions being deformable into the lumen; and a cap having a female thread for mating with the male thread, the female thread having a hole in communication with the lumen; wherein when the shaft is inserted into the hole and lumen and the female thread of the cap is threaded onto the male thread, the threaded portions deform into the lumen and shaft inserted therein and retain the shaft of the cotton swab in the cotton swab holding portion.

The holder can further comprise a container for holding one of a liquid or a gel disposed in one of the handle portion or cotton swab holding portion and dispensing means for dispensing the liquid or gel onto or into a cotton portion of the cotton swab. The container can comprise a deformable bladder having an orifice and the dispensing means can comprise a button disposed on one of the handle portion or cotton swab holding portion having an urging portion which pushes against the deformable bladder to dispense the liquid or gel from the orifice. The button can include a biasing member for biasing the button in a position in which the urging portion does not push against the deformable bladder.

The orifice can include a check valve which allows liquid or gel to flow from the deformable bladder onto or into the cotton portion of the cotton swab and prevents the liquid or gel from flowing into the deformable bladder.

The orifice can be in communication with a hollow portion of the shaft of the cotton swab when the shaft is retained in the cotton swab holding portion, the hollow portion being in communication with the cotton portion of the cotton swab.

Also provided is a cotton swab for use with a cotton swab holder. The cotton swab comprising: a shaft; and a cotton portion disposed on an end of the shaft; wherein the shaft has a length which when inserted into the cotton swab holder permits only substantially the cotton portion to extend from the cotton swab holder.

The shaft can include a hollow portion in communication with the cotton portion of the cotton swab.

The cotton portion can have a bulbous shape, a conical shape or a chisel shape. A leading edge of the chisel shape can be tapered.

Still further provided is a cotton swab and holder assembly. The assembly comprising: a cotton swab having a shaft and a cotton portion disposed on an end of the shaft; a handle portion; and a cotton swab holding portion connected to the handle portion, the cotton swab holding portion having a mechanism for holding the shaft of the cotton swab.

The shaft can have a length which when inserted into the cotton swab holding portion permits only substantially the cotton portion to extend from the cotton swab holding portion.

The mechanism can releasably retain a shaft of the cotton swab.

The handle portion can be an elongated handle portion adapted to be gripped by a hand of a user.

The cotton swab holding portion can be bulbous and have a proximal end connected to a distal end of the handle portion.

The mechanism for releasably retaining the cotton swab can comprise: a threaded shaft having a lumen, a male thread and two or more slits formed in the male thread so as to form threaded portions being deformable into the lumen; and a cap having a female thread for mating with the male thread, the female thread having a hole in communication with the lumen; wherein when the shaft is inserted into the hole and lumen and the female thread of the cap is threaded onto the male thread, the threaded portions deform into the lumen and shaft inserted therein and retain the shaft of the cotton swab in the cotton swab holding portion.

The assembly can further comprise a container for holding one of a liquid or a gel disposed in one of the handle portion or cotton swab holding portion and dispensing means for dispensing the liquid or gel onto or into the cotton portion of the cotton swab. The container can comprises a deformable bladder having an orifice and the dispensing means comprises a button disposed on one of the handle portion or cotton swab holding portion having a urging portion which pushes against the deformable bladder to dispense the liquid or gel from the orifice. The button can include a biasing member for biasing the button in a position in which the urging portion does not push against the deformable bladder. The orifice can include a check valve which allows liquid or gel to flow from the deformable bladder onto or into the cotton portion of the cotton swab and prevents the liquid or gel from flowing into the deformable bladder. The shaft can include a hollow portion in communication with the cotton portion of the cotton swab and the orifice is in communication with the hollow portion of the shaft when the shaft is retained in the cotton swab holding portion.

Still further yet provided is a cotton ball comprising: a cotton body; one or more walls defining a cavity disposed on an interior of the cotton body; and one of a liquid or gel contained in the cavity.

The one or more walls can comprise a thin film encapsulating the liquid or gel which can be burst upon application of pressure to the cotton body, thereby dispensing the liquid or gel from the cavity and into the cotton body.

The cotton ball can further comprise a piercing member disposed on the cotton body, the piercing member having a body and at least one projection wherein the body is pressed to force the projection to pierce the one or more walls, thereby dispensing the liquid or gel from the cavity and into the cotton body.

The cotton ball can further comprise a biasing means for biasing the projection away from the one or more walls.

The liquid or gel can be a medicament, a solvent or a cleaner.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates an isometric view of a cotton swab holder assembly.

FIG. 2 illustrates a side view of the cotton swab holder of FIG. 1.

FIG. 3 illustrates a section view of the cotton swab holder of FIG. 2 as taken along line 3-3 therein.

FIG. 4 illustrates an isometric exploded view of the cotton swab holder of FIG. 1.

FIGS. 5a-5c illustrate variations of cotton swabs for use in the cotton swab holder assembly of FIG. 1.

FIG. 6 illustrates a section view of a first embodiment of a cotton ball.

FIG. 7 illustrates an isometric side view of a second embodiment of a cotton ball.

FIG. 8 illustrates a side view of the cotton ball of FIG. 7.

### DETAILED DESCRIPTION OF THE PREFERRED EMBODIMENT

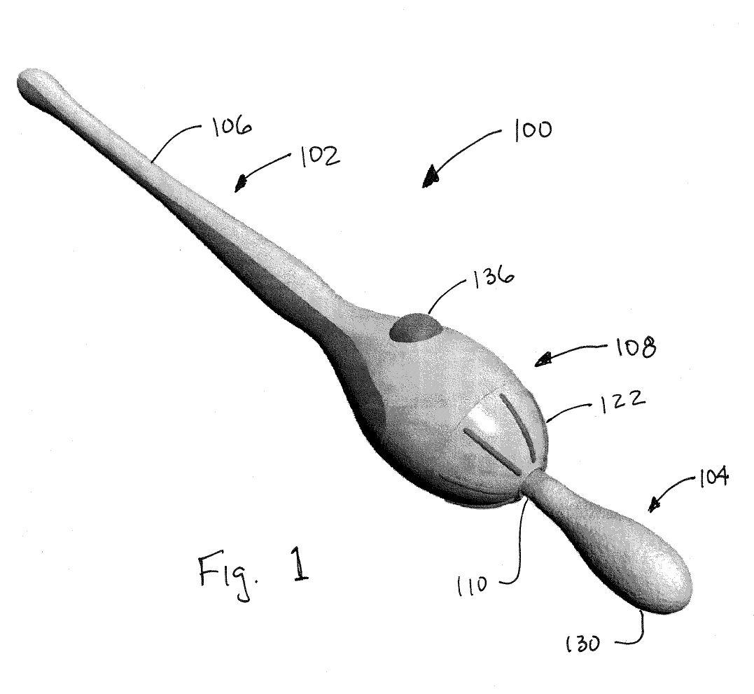

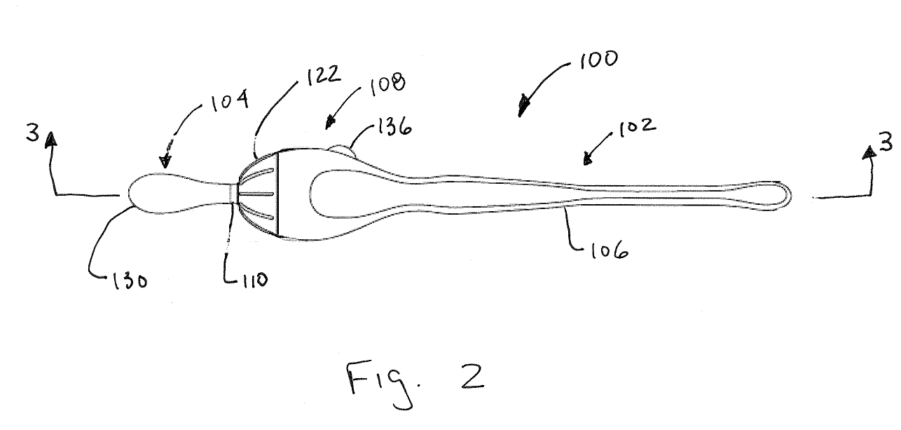

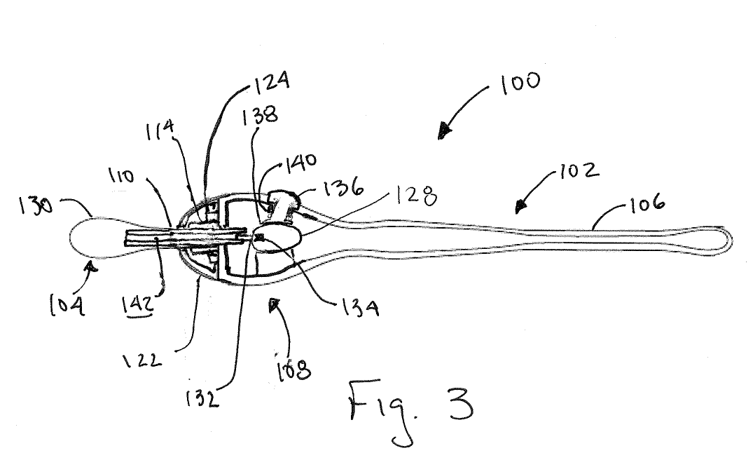

Referring now to FIGS. 1-3, there is shown a cotton swab holder assembly, generally referred to with reference numeral 100. The cotton swab holder assembly 100 includes a cotton swab holder 102 (hereinafter “holder”) and a cotton swab 104 retained therein.

The holder 102 can include a handle portion 106 and a cotton swab holding portion 108 connected to the handle portion 106. Although the handle portion 106 is shown integral with the cotton swab holding portion 108, the same can be of a two or multi-piece construction operatively connected to each other. Furthermore, the handle portion 106 is constructed so as to be adapted to be gripped by a hand of a user. As such, the handle portion 106 does not have to project from the cotton swab holding portion 108 and may merely be a proximal end 108a of the cotton swab holding portion 108. The cotton swab holding portion 108 can have any shape or size, such as a bulbous shape with a proximal end thereof connected to a distal end of the handle portion 108a of the cotton swab holding portion 108.

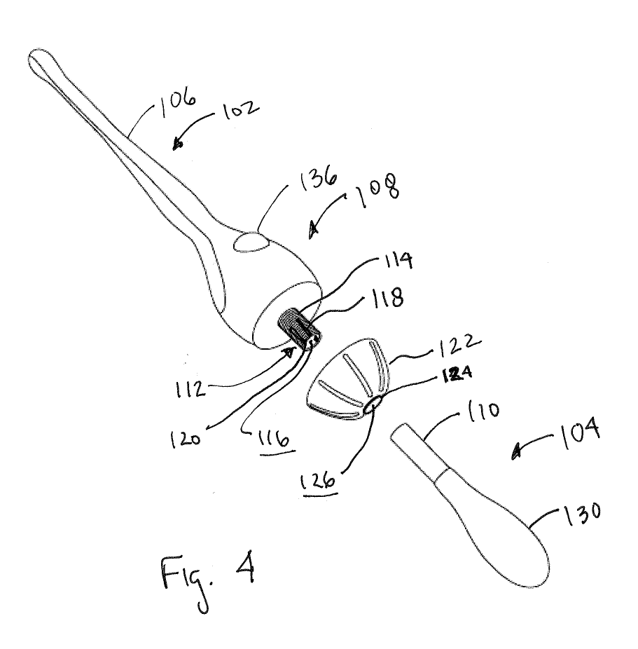

Referring additionally to FIG. 4, the cotton swab holding portion 108 includes a mechanism 112 for releasably retaining a shaft 110 of the cotton swab 104. The mechanism 112 can be any means known in the art for releasably retaining a projection, such as the shaft 110, therein. Such mechanism 112 can include a collet mechanism having a threaded shaft 114 with a lumen 116, where a male thread 118 and two or more slits 120 are formed in the male thread 118 so as to form threaded portions 120 that are deformable into the lumen 116. A cap 122 is provided which forms a portion of the cotton swab holding portion 108. The cap 122 has a female thread 124 for mating with the male thread 118 or the threaded shaft 114 and the female thread 124 has a hole 126 in communication with the lumen 116 of the threaded shaft 114 when the cap is threaded onto the male thread 118 of the threaded shaft 114.

When the shaft 110 is inserted into the hole 126 and lumen 116 and the female thread 124 of the cap 122 is threaded onto the male thread 118. The threaded portions 120 deform into the lumen 116 and the shaft 110 inserted therein to retain the shaft 110 of the cotton swab 104 in the cotton swab holding portion 108. Such collet mechanisms are well known in the art of machinery.

Other types of mechanisms for retaining the shaft 110 in the cotton swab holding portion 108 can also be used, such as a spring loaded detent system having one or more detents on one of the shaft 110 or lumen 116 and the other having one or more mating detents. Alternatively, one or more movable jaws that grip the shaft 110 can be provided, where the jaws are operable between positions that grip the shaft 110 or allow the shaft 110 to be released. Similarly, an offset cam can be used, such that when the same is rotated, a surface of the cam is urged against the shaft 110 to retain the same and when the cam is either further rotated or rotated in an opposite direction, the surface is released from being urged against the shaft 110 to allow the shaft 110 to be removed.

The holder 100 can also include a container 128 for holding one of a liquid or a gel disposed in the handle portion 106 or cotton swab holding portion 108. The container 128 also includes a dispensing means for dispensing the liquid or gel onto or into a cotton portion 130 of the cotton swab 104. The container 128 can be formed of any material and in any shape known in the art, such as a deformable bladder having an orifice 132. The orifice can include a check valve 134, such as a duckbill type valve known in the art which allows the liquid or gel in the container 128 to flow from the deformable bladder onto or into the cotton portion 130 of the cotton swab 104 and prevents the liquid or gel from flowing back into the deformable bladder. Such check valve can also close when pressure is no longer applied to the container 128. The dispensing means can also include a button 136 disposed on the handle portion 106 or cotton swab holding portion 108 having an urging portion 138 which pushes against the deformable bladder 128 to force the liquid or gel from the orifice 132. The button includes a biasing member 140, such as a spring for biasing the button 136 in a position in which the urging portion 138 does not push against the deformable bladder. The orifice 132 is in communication with a hollow portion 142 of the shaft 110 of the cotton swab 104 when the shaft is retained in the cotton swab holding portion 108 and the hollow portion 142 is in communication with the cotton portion 130 of the cotton swab 104. Thus, depressing the button 136 causes the urging portion 138 to deform the deformable bladder which forces liquid or gel disposed therein to flow from the orifice 132. The liquid or gel flowing through the orifice 132 continues through the hollow portion 142 of the shaft 110 and into the cotton portion 130 of the cotton swab 104.

When the container 128 is empty, the holder 100 can be discarded or used without any liquid or gel. Alternatively, the container can be replaced in the holder with a container having the same or a different liquid or gel disposed therein.

The liquid or gel can have any of medical, household, cosmetic or industrial uses. For example, such liquid or gel can be a medicament, such as hydrogen peroxide or an ointment that is to be applied to a wound, a solvent, such as nail polish remover, to be applied to a surface or an industrial cleaner applied to a surface.

Of course, other dispensing means can also be used, such as a pump housed in the handle portion 106 of the cotton swab holding portion 108 which is actuated by an electrical switch (which can be opened/closed by the actuation of the button 136) to deliver the liquid or gel through the hollow portion 142 to the cotton portion 130 of the cotton swab 104. Such liquid or gel can also be applied externally to the cotton portion 130, such as by being sprayed thereon by a pump or manually applied pressure.

Referring back to FIGS. 1 and 4, there is shown a cotton swab 104 for use with the cotton swab holder 100. As discussed above, the cotton swab 104 includes a shaft 100 and a cotton portion 130 disposed on an end of the shaft 100. The shaft 110 can have any length, however, to avoid ear injuries, the length can be as such that when it is inserted into the cotton swab holder 100, permits only substantially the cotton portion 130 to extend from the cotton swab holder 100. As such, the cotton portion 130 cannot extend to the sensitive portions of the ear. As also discussed above, the shaft 110 can include a hollow portion 142 in communication with the cotton portion 130 of the cotton swab 104 to facilitate delivery of the liquid or gel from the container 128.

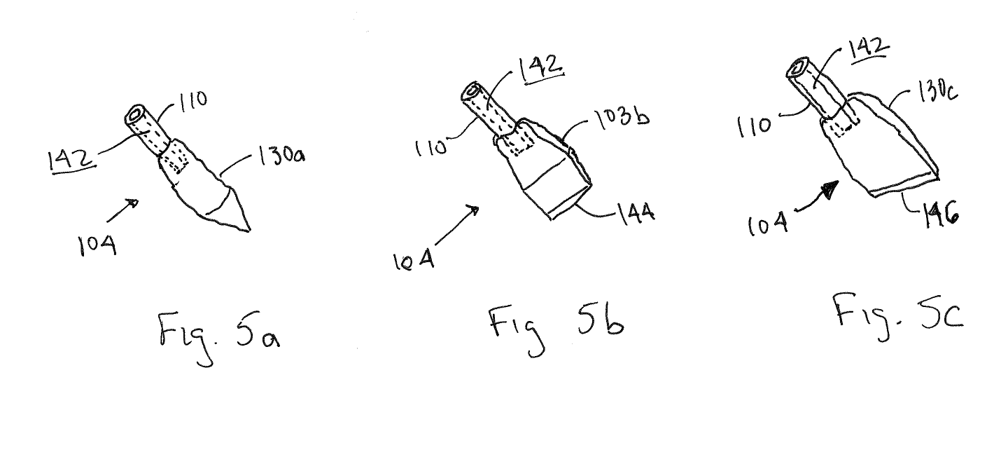

As shown in FIGS. 1 and 4, the cotton portion 130 can have a bulbous shape. However, other shapes are possible, which may be more functional or useful for particular applications. For example, as shown in FIG. 5a, the cotton portion 130a can have a conical shape having a pointed end (or truncated point). As shown in FIG. 5b, the cotton portion 130b can have a chisel shape where the leading edge 144 of the chisel shape can be perpendicular to the shaft 110 or tapered 146 as shown in FIG. 5c.

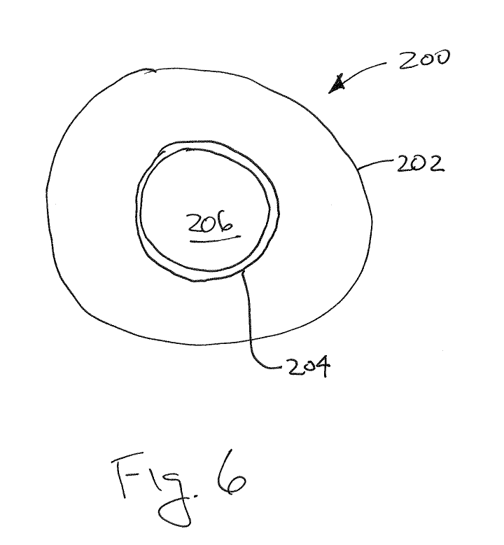

Referring now to FIG. 6, there is illustrated therein a cotton ball, generally referred to by reference numeral 200. Although such cotton ball 200 is referred to as a “ball,” the shape thereof does not have to be spherical but can be of any shape known in the art. The cotton ball 200 has a cotton body 202 and a wall 204 defining a cavity 206 disposed on an interior of the cotton body 202. Although the wall 204 is illustrated and described as being totally interior of the cotton body 202, a portion or some portions of the wall 204 can extend to an exterior of the cotton body 202. A liquid or gel is contained in the cavity. Like the cavity described above with regard to the cotton swab holder, the liquid or gel can have any of medical, household, cosmetic or industrial uses. For example, such liquid or gel can be hydrogen peroxide or ointment that is to be applied to a wound, to a solvent, such as nail polish remover, to be applied to a surface or an industrial cleaner applied to a surface.

The wall 204 can be a single spherical or other enclosed wall or a series of two or more walls defining a closed cavity 206. The wall(s) 204 can comprise a thin film which encapsulates the liquid or gel and which can be easily burst upon application of pressure to the cotton body, thereby dispensing the liquid or gel from the cavity 206 and into the cotton body 202. The wall(s) 204 can also have one or more orifices (not shown) which are opened to dispense the liquid or gel upon pressure to the cotton body 202 and which at least partially close upon removal of such pressure to allow control of the amount of liquid or gel that is provided to the cotton body 202. As another alternative, the orifice can have a small plug (not shown) that is connected to a strip (not shown) that extends to or above a surface of the cotton body 202 such that when the strip is pulled, the strip removes the plug from the orifice and allows the liquid or gel to flow into the cotton body 202. Such orifices can also have check valves (not shown) such as duckbill type valves known in the art, for closing the orifices when the pressure is removed and/or to prevent back-flow of the liquid or gel into the cavity 206.

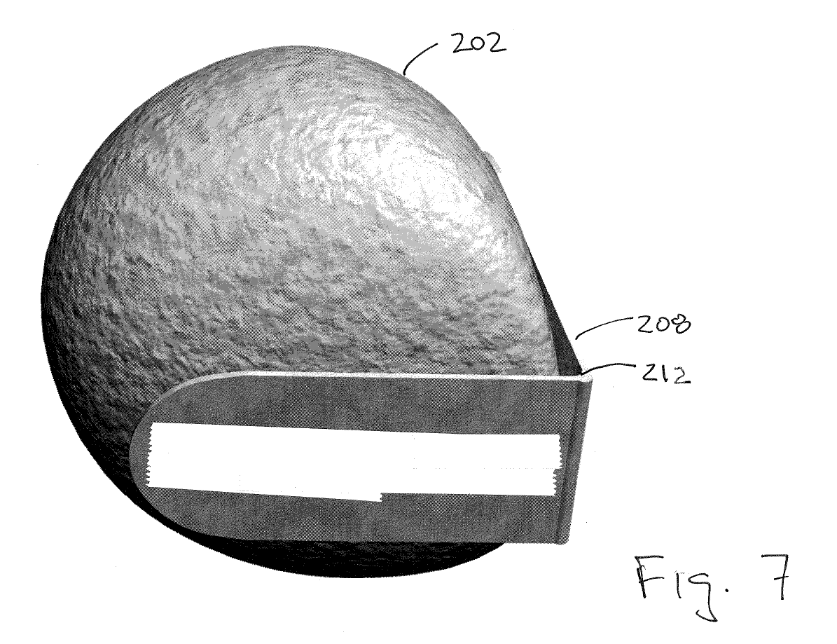

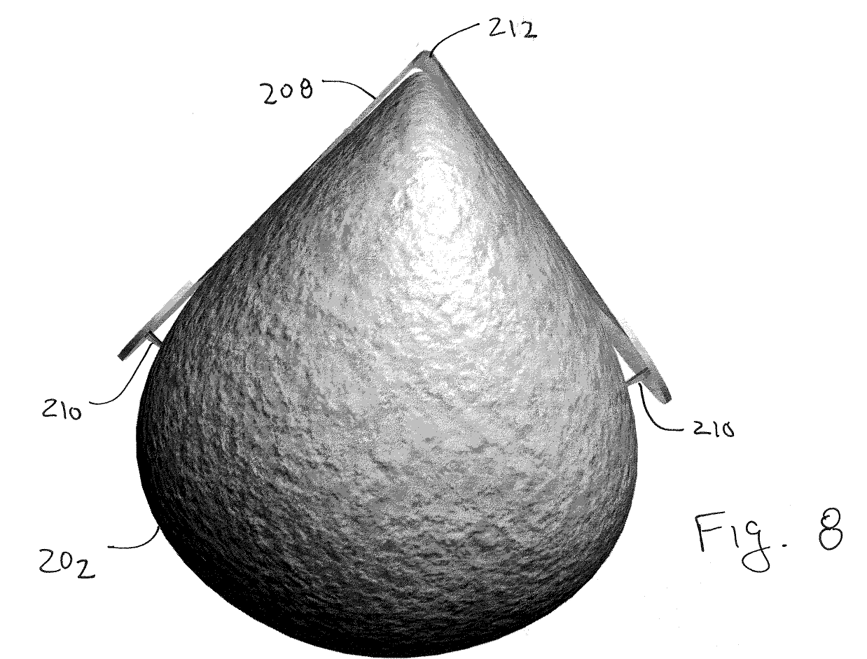

Referring now to FIGS. 7 and 8, alternatively, a piercing member 208 may be provided and disposed on the cotton body 202. The piercing member 208 having a body, which can be V- or U-shaped to extend over a portion of the cotton body 202 and at least one projection 210 disposed such that a force exerted on the body causes the one or more projections 210 to pierce the wall(s) 204, thereby dispensing the liquid or gel from the cavity 206 and into the cotton body 202. The one or more projections 210 of the piercing member 208 can be biased away from the wall(s) 204, such as by use of a resilient material for the piercing member 208. In the case where the piercing member 208 is V- or U-shaped, at least an apex 212 of the piercing member 208 can be formed of the resilient material to act as a spring-loaded hinge and bias the projections 210 away from the wall(s) 204. The piercing member 208 can also act as a holding portion for the user to grasp while using the cotton ball 200 so as to avoid contact with the fluid or gel that saturates the cotton body 202.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A holder for a cotton swab, the holder comprising:
a handle portion; and
a cotton swab holding portion connected to the handle portion, the cotton swab holding portion having a mechanism for releasably retaining a shaft of the cotton swab.

2. The holder of claim 1, wherein the handle portion is an elongated handle portion adapted to be gripped by a hand of a user.

3. The holder of claim 1, wherein the cotton swab holding portion is bulbous and has a proximal end connected to a distal end of the handle portion.

4. The holder of claim 1, wherein the mechanism for releasably retaining the cotton swab comprises:
a threaded shaft having a lumen, a male thread and two or more slits formed in the male thread so as to form threaded portions being deformable into the lumen; and
a cap having a female thread for mating with the male thread, the female thread having a hole in communication with the lumen;
wherein when the shaft is inserted into the hole and lumen and the female thread of the cap is threaded onto the male thread, the threaded portions deform into the lumen and shaft inserted therein and retain the shaft of the cotton swab in the cotton swab holding portion.

5. The holder of claim 1, further comprising a container for holding one of a liquid or a gel disposed in one of the handle portion or cotton swab holding portion and dispensing means for dispensing the liquid or gel onto or into a cotton portion of the cotton swab.

6. The holder of claim 5, wherein the container comprises a deformable bladder having an orifice and the dispensing means comprises a button disposed on one of the handle portion or cotton swab holding portion having an urging portion which pushes against the deformable bladder to dispense the liquid or gel from the orifice.

7. The holder of claim 6, wherein the button includes a biasing member for biasing the button in a position in which the urging portion does not push against the deformable bladder.

8. The holder of claim 5, wherein the orifice includes a check valve which allows liquid or gel to flow from the deformable bladder onto or into the cotton portion of the cotton swab and prevents the liquid or gel from flowing into the deformable bladder.

9. The holder of claim 8, wherein the orifice is in communication with a hollow portion of the shaft of the cotton swab when the shaft is retained in the cotton swab holding portion, the hollow portion being in communication with the cotton portion of the cotton swab.

10. A cotton swab for use with a cotton swab holder, the cotton swab comprising:
a shaft; and
a cotton portion disposed on an end of the shaft;
wherein the shaft has a length which when inserted into the cotton swab holder permits only substantially the cotton portion to extend from the cotton swab holder.

11. The cotton swab of claim 10, wherein the shaft includes a hollow portion in communication with the cotton portion of the cotton swab.

12. The cotton swab of claim 10, wherein the cotton portion has a bulbous shape.

13. The cotton swab of claim 10, wherein the cotton portion has a conical shape.

14. The cotton swab of claim 10, wherein the cotton portion has a chisel shape.

15. The cotton swab of claim 14, wherein a leading edge of the chisel shape is tapered.

16. A cotton swab and holder assembly, the assembly comprising:
a cotton swab having a shaft and a cotton portion disposed on an end of the shaft;
a handle portion; and
a cotton swab holding portion connected to the handle portion, the cotton swab holding portion having a mechanism for holding the shaft of the cotton swab.

17. The assembly of claim 16, wherein the shaft has a length which when inserted into the cotton swab holding portion permits only substantially the cotton portion to extend from the cotton swab holding portion.

18. The assembly of claim 16, wherein the mechanism releasably retains a shaft of the cotton swab.

19. The assembly of claim 16, wherein the handle portion is an elongated handle portion adapted to be gripped by a hand of a user.

20. The holder of assembly of claim 16, wherein the cotton swab holding portion is bulbous and has a proximal end connected to a distal end of the handle portion.

21. The assembly of claim 16, wherein the mechanism for releasably retaining the cotton swab comprises:
a shaft having a lumen, a male thread and two or more slits formed in the male thread so as to form threaded portions being deformable into the lumen; and
a cap having a female thread for mating with the male thread, the female thread having a hole in communication with the lumen;
wherein when the shaft is inserted into the hole and lumen and the female thread of the cap is threaded onto the male thread, the threaded portions deform into the lumen and shaft inserted therein and retain the shaft of the cotton swab in the cotton swab holding portion.

22. The assembly of claim 16, further comprising a container for holder one of a liquid or a gel disposed in one of the handle portion or cotton swab holding portion and dispensing means for dispensing the liquid or gel onto or into the cotton portion of the cotton swab.

23. The assembly of claim 22, wherein the container comprises a deformable bladder having an orifice and the dispensing means comprises a button disposed on one of the handle portion or cotton swab holding portion having a urging portion which pushes against the deformable bladder to dispense the liquid or gel from the orifice.

24. The assembly of claim 23, wherein the button includes a biasing member for biasing the button in a position in which the urging portion does not push against the deformable bladder.

25. The assembly of claim 23, wherein the orifice includes a check valve which allows liquid or gel to flow from the deformable bladder onto or into the cotton portion of the cotton swab and prevents the liquid or gel from flowing into the deformable bladder.

26. The assembly of claim 25, wherein the shaft includes a hollow portion in communication with the cotton portion of the cotton swab and the orifice is in communication with the hollow portion of the shaft when the shaft is retained in the cotton swab holding portion.

27. A cotton ball comprising:
a cotton body;
one or more walls defining a cavity disposed on an interior of the cotton body; and
one of a liquid or gel contained in the cavity.

28. The cotton ball of claim 27, wherein the one or more walls comprise a thin film encapsulating the liquid or gel which can be burst upon application of pressure to the cotton body, thereby dispensing the liquid or gel from the cavity and into the cotton body.

29. The cotton ball of claim 27, further comprising a piercing member disposed on the cotton body, the piercing member having a body and at least one projection wherein the body is pressed to force the projection to pierce the one or more walls, thereby dispensing the liquid or gel from the cavity and into the cotton body.

30. The cotton ball of claim 27, further comprising a biasing means for biasing the projection away from the one or more walls.

31. The cotton ball of claim 27, wherein the liquid or gel is a medicament.

32. The cotton ball of claim 27, wherein the liquid or gel is a solvent.

33. The cotton ball of claim 27, wherein the liquid or gel is a cleaner.
